(19) United States
(12) Patent Application Publication
(10) Pub. No.: US 2009/0098230 A1
(43) Pub. Date: Apr. 16, 2009

(54) NUTRACEUTICAL MORINGA COMPOSITION

(76) Inventors: David A. Andrews, Draper, UT (US); Kathleen Andrews, Draper, UT (US)

Correspondence Address: BATEMAN IP LAW GROUP, P.O. BOX 1319, SALT LAKE CITY, UT, 84110, US

(21) Appl. No.: 12/338,789

(22) Filed: Dec. 18, 2008

Related U.S. Application Data
(62) Division of application No. 11/384,213, filed on Mar. 16, 2006.
(60) Provisional application No. 60/663,026, filed on Mar. 18, 2005.

(51) Int. Cl. A61K 36/00

(52) U.S. Cl. 424/774

(57) ABSTRACT

The present invention incorporates portions from the Moringa plant into a nutraceutical beverage to provide a therapeutic effect in a person. The leaves, seeds, and fruit of the Moringa plant are used in the present invention to provide a biologically metabolized nutritional composition for health, well-being, and for treatment of ailments.

### RELATED APPLICATIONS

The present application claims the benefit of U.S. Provisional Patent Application No. 60/663,026, filed Mar. 18, 2005, which is incorporated herein in its entirety.

### BACKGROUND OF THE INVENTION

2. The Field of the Invention

The present invention relates to the field of dietary supplements, nutritional supplements, pharmaceutical preparations, nutritious foods and beverages. More specifically, the invention relates to nutraceutical compositions utilizing the leaves, the seeds and the fruits of the Moringaceae Dumort plant family for a variety of nutritional medicinal purposes.

2. State of the Art

The Moringaceae Dumort is a mono-generic genus plant family with 13 known species: Moringa arborea, Moringa borziana, Moringa concanensis, Moringa drouhardii, Moringa hildebrandtii, Moringa longituba, Moringa oleifera, Moringa ovalifolia, Moringa peregrina, Moringa pygmaea, Moringa rivae, Moringa ruspoliana and Moringa stenopetala. Of these 13 species, Moringa oleifera Lamarck (synonymous with Moringa pterygosperma C.F. Gaertn.) is the most widely known and utilized species. The Moringa oleifera L. was named by Swedish biologist Carl Linaeus in the 1700s, and is a tree species native to and still found wild in the sub-Himalayan tract of the Indo-Pakistan subcontinent. The Moringa species are located in Africa, Arabia, Southeast Asia, the Pacific and Caribbean Islands, South America and now in all other tropical and sub-tropical parts of the world where it easily takes root.

The Moringa genus plant family and the Moringa oleifera L., in particular, has deciduous foliage or leaflets that are one to two centimeters in diameter, in a pale to dark green color. The pleasantly fragranced flowers are generally yellow in color except that the upper slender stem that supports the blade of the foliage or flower is cream to white in color. The fruits are drumstick-shaped pods that begin their rapid growth slim and tender with a light green color. As the fruits mature, they become dark green, three sided or nearly cylindrical, nine-ribbed, pointed at the apex, tapered at the base, brown, thinly woody, triangular shaped and up to 24 inches long. The plant's seeds are enclosed within the fruit pods. The seeds are oily, round, or triangular shaped with the kernel surrounded by a lightly wooded shell or husk, composed of three papery wings.

The Moringa family genus of trees is also known as the Horseradish trees (named by English settlers due to the taste of the condiment prepared from the roots) or Drumstick trees (due to the shape of the pods being much like sticks utilized to play the drums). Other cultures and languages have also named the tree with specific vernacular names like Ben Oil Tree or Ben Tree, Shigru or Sigru, Kelor, Moringo, Sahjan or Soanjan, Mullangay and even the Miracle Tree.

The Moringa is a deciduous plant that grows in tree stands and generates new growth/shoots with leaf-bearing branches as fast as nine feet in a 10 month period. Its mature height varies from 15 to 40 feet and it has open, umbrella-shaped crowns with a straight trunk that is four to 12 inches in diameter with corky, whitish bark. The branches are slender, wide-spreading, drooping and fragile. The plant, when grown from a seed, is characterized by a tuberous tap root which is deeply imbedded in the soil, helping to explain the tree's documented tolerance of arid or drought conditions. Although it appears to prefer well-drained sandy soils, Moringa can grow in many other soil conditions including some clay soils. The tree tolerates light frosts and can also grow in slightly alkaline soils with a pH up to 9.

In the traditions of Ayurvedic medicine from India, the Moringa plant was first described around the first century AD in Vedic (translated to mean “wisdom” or “knowledge”) scriptures, where the prophet/physician Dhavantari revealed to his disciples that the leaves and fruit were nutritious and that the seed oil has curative properties. Ayurvedic medical treatments have existed for over five thousand years. Ayurveda comes from two Sanskrit words: Ayu means “life” and Veda means “knowledge of”. In essence, this Science of Life creates harmony with nature and uses Mother Nature to create health and balance within the human body. Ayurvedic medicine teaches using natural plants to promote self healing, good health and longevity, and have declared that Moringa can provide the nutrients and therapeutic ingredients to prevent, mitigate or treat many diseases or conditions. The plant is known to have a number of beneficial uses as a highly nutritious food, for medicinal purposes, for water purification and even as a fast growing windbreak or fence line.

Moringa seed oil is used as a natural antibiotic and antifungal, as well as to treat liver and stomach disorders. Moringa leaves and seeds contain 4-(α-L-rhamnopyranosyloxy)-benzylgucosinolate and three monoacetyl isomers of this glucosinate, and treat diarrhea, regulate blood sugar, normalize blood cholesterol and are reputed to be aphrodisiac. Moringa leaf juice is used as a diuretic and as a skin antiseptic. Its flowers and leaves are both anti-helminitic and commonly used internally and externally as a poultice against parasites. The leaf stems and plant bark are used to cure eye diseases and treat scurvy, sores and skin infections. The plant roots are used as a stimulant against paralytic attacks, to combat intermittent fevers and to relieve chronic rheumatism.

The Moringa seeds are used for treating abdominal tumors, hysteria, bladder and prostate troubles. The seeds contain a number of potent antioxidants that contribute to the oil's stability, antioxidant qualities and non-rancidity. In addition, the Moringa seeds contain tocopherols (vitamin E), antioxidants that are known to have protective qualities against pollutants such as cigarette smoke and even chemical smog. Moringa's fruits have been shown to have an inhibitory effect against the growth of a number of tumor types without any general host toxicity. The Moringa plant has additional medicinal usage as a cardiac and circulatory stimulant. The seeds of this plant also have a purifying effect by flocculation on turbid water.

The leaves, seeds and fruits have great nutritional values proven to overcome the many effects of malnutrition for both children and adults. The seeds, when harvested from the fruit pods, can be eaten like peas or even roasted like peanuts. The leaves are used in vegetable curries, while the unripe fruit pods can be pickled, used in curry, or boiled and sliced like green beans. The fruit pods taste similar to asparagus and are highly nutritious. The fruit pods are used in Indian cuisine as a vegetable, as well as in Indian soups, sauces and curries. When cooked, the nutritious flowers are reportedly similar in taste to mushrooms. The root of the tree may be shredded and used as a condiment which has a similar taste to horseradish. The crushed seeds yield the Oil of Ben/Behen or behenic acid (C22:0) that is used as a salad oil. The oil, first identified in 1847, is also used in cooking as it resists spoiling and rancidity.

In addition, the high-quality oil from the Moringa is used in soaps, perfume, hair oil, other cosmetics and in artist's paints, as well as a lubricant for machinery and other industrial uses. Moringa seeds have been used to purify water by flocculation to replace expensive and toxic conventional and industrial chemicals such as aluminum sulfate commonly used in water treatment plants. The sap or gum from the Moringa wood that exudes from incisions, is insoluble in water and has been used in printing calico and tanning leather or to make dye. The timber is suited for pulp production to make cellophane or rayon. Moringa seed cake (compressed seeds) remains after the Oil of Ben has been largely removed. Moringa seed cake has been used as a ground fertilizer or cattle feed, but generally this by-product of the oil extraction process is merely discarded. The Moringa seed cake has not been previously used in a nutraceutical.

The Moringa leaves, fruits and seeds contain mustard oil glycosides (niazinin, niazimicin, niaziminin and isothiocyanate) which have a hypotensive activity. In addition, niazimicin has been reported to be a potent anti-tumor agent. The highly nutritious leaves are a significant source of beta-carotene (vitamin A), vitamin C, proteins, calcium, iron and potassium. The plant leaves contain cytokinins in the form of zeatin as well as other beneficial phytochemicals such as vanillin, beta-sitosterol, caffeoylquinic acids, kaempferol, quercetin, octacosanoic acid, moringine, moringinine, bayrenol, indole acetic acid, indole acetonitrile, benzylisothiocyanate, pterygospermine and carotenes. The leaves also contribute great values of calcium, magnesium, phosphorus, potassium, sulfur, manganese, zinc, selenium, vitamin E, vitamin B2 (riboflavin), vitamin B3 (niacin), choline, alanine, aspartic acid, glutamatic acid glycine, histidine, isoleucine, leucine, lysine, methionine, proline, serine, threonine, tryptophan, tyrosine, valine, and chlorophyll. The flowers and leaves contain the antioxidants quercetin and kaempferol. Moringa seeds contain large amounts of iron, vitamin B1 (thiamin), the amino acids: arginine, cysteine and phenylalanine and fats including essential omega-3 oils. The Moringa fruits and pods contain substantial amounts of the essential mineral copper as well as vitamin C, vitamin A and sodium. The essential fatty acids (omega-3, omega-6 and omega-9) are all found in the Moringa plant. It is believed that the essential fatty acids work synergistically to protect the cell integrity and general physiology of the body, when all three are present in efficacious amounts. It is interesting to note that the leaves contain generous amounts of the omega-3 and 6 oils and only traces of the omega-9 oil, while the seeds contain generous amounts of the omega-9 oil and just trace elements of the omega-3 and 6 oils. The efficacious combination of the leaves and seeds further enhances the efficacy of the essential fatty acids.

Naturally occurring within different parts of the Moringa plant are several important active ingredients. Chlorophyll is the green pigment or life blood of Moringa. The chlorophyll molecule contains magnesium and is believed to strengthen the blood and support a healthy mouth, stomach and intestinal tract by countering bad breath, ulcers and internal wounds. Other studies have shown that chlorophyll may cure acute infections of the respiratory tract and sinuses, as well as, filter any cancer-inducing environmental toxins found in the air or food, that humans may breathe or ingest. Finally, chlorophyll supports normal liver function and detoxification of the body even from the effects of alcohol. Cytokinins are small molecules that act as plant hormone compounds and are found in higher plants. Cytokinins are similar in structure to adenine (a major component of the genetic material in the cell). Cytokinins stimulate cell division and the growing of tissues. The cytokinins found so far in the Moringa plant have powerful anti-aging effects on the human body and skin. Cytokinins delay the process of aging, death and cell deterioration in plants and promote nutrient uptake. The application of a cytokinin to the leaf of an aging plant actually promotes the leaf to stay green longer, while other surrounding leaves, without the cytokinin application will yellow and die. The present invention discloses that Moringa trees under two years of age contain beneficial high amounts of chlorophyll, while mature Moringa trees contain beneficial high amounts of cytokinins. By combining an efficacious amount of both mature and young Moringa plants, chlorophyll and cytokinins are obtained in the most beneficial amounts and believed to work synergistically to provide the most efficacious nutraceutical composition.

The cytokinins identified to date in Moringa (zeatin, dihydrozeatin and isopentyladenine) are found mainly in the leaves; however, the seeds are also a significant source of the cytokinin, dihydrozeatin. The cytokinins act synergistically when the seed and leaf components are blended together as described herein, enhancing the anti-aging, antioxidant and cell protective functions.

Zeatin is the name of the first naturally occurring cytokinin that was isolated in 1961. Moringa has tremendous quantities of zeatin; one of the most potent cytokinins discovered, so far, that affects the rate of aging. The greatest concentration of zeatin is in the Moringa leaves, but it is also present in substantial amounts in the seeds. Zeatin delays aging by its influence on cell division and through its antioxidant properties. Studies have confirmed that zeatin protects humans against neuronal toxicity onset with age.

Quercetin is a plant pigment or flavonoid found in the Moringa leaf that acts as an antihistaminic, reduces inflammation associated with some forms of arthritis, and is also a powerful antioxidant that works to protect the cells from damaging particles known as free radicals. Quercetin is located in the leaves as well as in the seeds and fruit pods. There are indications that quercetin may be helpful in preventing the absorption of excess cholesterol and also in protecting cells against mutations.

Kaempferol is a flavonoid related to quercetin with additional powerful antioxidant properties. Kaempferol is located in the leaves, seeds and fruit pods. It has been shown to inhibit the growth of tumor cells.

Beta-sitosterol is a plant sterol, or hormone, and the main component of a group of plant sterols called phytosterols. Beta-sitosterol has been located so far only in the leaves of the Moringa plant. Its healthy chemical structure is very similar to cholesterol, thus substituting itself for cholesterol and preventing some actual cholesterol from being absorbed by the body. Beta-sitosterol helps boost the immune system as well.

Caffeoylquinic acids are also found in the Moringa leaves, fruit pods and seeds. These antioxidant acids provide specific hepatoprotector effects by increasing bile production and reducing plasma cholesterol. Caffeoylquinic acids reduce the symptoms of abdominal pain, bloating, lack of appetite and nausea associated with liver and digestive disorders.

Moringa contains all essential amino acids along with many others, namely, aspartic acid, glutamic acid, serine, glycine, threonine, alanine, valine, leucine, isoleucine, histidine, lysine, arginine, phenylalanine, tryptophan, cystine, serine, proline, tyrosine and methionine. Amino acids are the building blocks of proteins and many amino acids have antioxidant and anti-inflammatory effects. The combination of multiple antioxidants and/or anti-inflammatory agents appears to have a synergistic effect in the body, with increased potency and effectiveness. Some amino acids are only found in the Moringa seeds, while other amino acids are found exclusively in the leaves of the Moringa plant. The efficacious combination of the leaves and seeds of the Moringa plant to provide the most beneficial blend of synergistic amino acids, is not found in any prior art.

However, despite the pharmacological and nutritional benefits of individual cytokinins, amino acids, and other compounds and the indigenous medicinal uses of the leaves, fruits and seeds of the Moringa plant, a nutraceutical composition containing the holistic benefits of the entire Moringa plant, including the leaves, fruits and seeds, is not known. Rather, Moringa is only used in traditional forms and those individuals using a particular plant component never receive the complete health benefits of this remarkable plant.

There exists a need in the nutraceutical arts for nutraceutical compositions that offer the health benefits of the whole Moringa plant, including leaves, fruits and seeds. There also exists a need for a nutraceutical composition rich in natural cytokinins, such as zeatin and dihydrozeatin, as well as quercetin, kaempferol, caffeoylquinic acid, beta-sitosterol, amino acids, essential fatty acids, vitamins and minerals for the treatment of a variety of human ailments, deficiencies and diseases in an efficacious manner. Further, there is a need in the art for a natural product containing the aforementioned ingredients and qualities that is economical to manufacture.

### SUMMARY OF THE INVENTION

The present invention relates to nutraceutical compositions derived from the leaves, and/or fruits, and/or seeds of the Moringa species plant. In particular, it is an object of the present invention to provide a nutraceutical composition rich in beta-sitosterol, caffeoylquinnic acid, kaempferol, quercetin and zeatin derived from the leaves, fruits and seeds of the Moringa plant, without the adverse side effects associated with synthetic ingredients. This is advantageous, as these natural ingredients have great health benefits and when combined in an efficacious amount, are believed to act together as the trigger to rapid and complete metabolism in humans. It is also believed that, in accordance with the present invention, amino acids and essential omega oils, some coming nearly exclusively from the seed cake and others primarily from the leaves, also participate in this action to trigger the metabolism.

In addition, an object of the present invention is to provide a method of preparing nutraceutical compositions of the Moringa plant species components that yields efficacious health supplements rich in natural cytokinins such as, zeatin and dihydrozeatin.

Another object of the present invention is to provide nutraceutical compositions rich in vital amino acids and essential fatty acids from the proper combination of the Moringa plant components.

It is a further object of the present invention to provide a nutraceutical composition that contributes to general human wellness and good health through the inclusion of the Moringa plant in a novel mixture comprising the leaves, fruits, fruit juice, seeds and seed cake.

It is a further object of the present invention to heighten the biological availability of the Moringa plant nutrients by combining the various Moringa plant components that include the natural beta-sitosterol, caffeoylquinic acid, kaempferol, quercetin and zeatin, along with essential fatty acids and amino acids in order to achieve a metabolic trigger that promotes rapid and increased absorption of the nutrients. The biologically-available nutrients are more easily absorbed, promoting more effective metabolism, producing energy and improved organ and tissue health. This combination of plant ingredients comprising the metabolic trigger, is more easily taken into the body when introduced with the addition of selected fruit or vegetable juices and juice extracts that enhance the rapid digestion and taste of the composition.

It is a further object of the present invention to provide an antimicrobial and anti-inflammatory composition resulting from mixed compounds of the Moringa plant species.

It will be appreciated that not all compositions made in accordance with the principles of the present invention include all objects of the invention, and said objects should not be viewed as narrowing the appended claims. The above-described features and advantages of the present invention, as well as additional features and advantages, will be set forth or will become more fully apparent in the description that follows and in the claims. Furthermore, the features and advantages of the invention may be learned by the practice of the invention, or will be obvious to one skilled in the art from the description, as set forth hereinafter. While the methods and processes of the present invention have proven to be particularly useful in the area of nutritional health supplements, those skilled in the art can appreciate that the methods and processes can be used in a variety of different applications and in a variety of different areas of manufacture to satisfy a wide-ranging variety of pharmaceutical and medicinal needs.

### DETAILED DESCRIPTION

The present invention relates to nutraceutical compositions derived from the Moringa species of plants. In particular the compositions of the invention, described herein, uniquely provide the naturally-occurring beta-sitosterol, caffeoylquinic acid, kaempferol, quercetin, zeatin, amino acids and essential fatty acid compounds from the leaves, seeds, and fruits of the Moringa plants. The invention also relates to processes of manufacturing the nutraceutical compositions described herein, in an economical manner.

There is no prior art reference that teaches the combination of the fruits, seeds or leaves of the Moringa plant into a nutraceutical. The present compositions of this invention are not obvious, known or recognized because of several factors: 1) the fruit and fruit juice have a particularly pungent smell that does not suggest combination with any other plant component, or incorporation into a nutraceutical composition, and especially not into a beverage; 2) the seeds are so oily that if combined with the juice, or any juice or other water based liquid, the mixture would never fully blend, due to the excess oil; and 3) the seed cake component of the Moringa plant has never been used as a nutraceutical and there is no prior art that it was ever used to treat any ailments or provide nutrition to humans. The benefits of the seed cake are not known and it is discarded after the Ben oil extraction process, or it is merely used as fertilizer and/or animal fodder.

It is understood that the terminology utilized herein, is used for the purpose of describing particular embodiments only, and is not intended to limit the scope of the present invention. In the disclosure and in the claims, the term “nutraceutical” shall refer to any compounds, chemicals or combinations that can provide medical, therapeutic, dietary or health benefits when consumed by humans or animals.

Unless defined otherwise, all technical and scientific terms used herein, have the same meanings as commonly understood by one of ordinary skill in the art to which this invention belongs. Preferred methods, compositions and materials of the present invention are described herein, although any methods and materials similar or equivalent to those described herein, can be used in the practice or testing of the present invention.

When used in the claims, “a portion” is meant to include all portions of the plant component. For example, “a leaf portion” is to include the actual leaf, whether ground or otherwise processed, along with any extracts derived from the leaf. In addition, “a portion of the seed” is meant to encompass the seed, or any extract from the seed such as the oil, and the seed cake. The terms “a portion of the fruit” is meant to encompass the fruit, the fruit pods, and the fruit juice, in any combination thereof.

Pharmacological and botanical researchers have discovered that the nutritional, bioavailable and medicinal properties of the Moringa leaves, seeds and fruits can be attributed in part to the naturally occurring cytokinins, antioxidants, amino acids, vitamins, minerals and other natural compounds contained in the plant.

Recent research has shown that the zeatin compound, a natural cytokinin, is found in great abundance in Moringa , along with other cytokinins. The natural constituents of this cytokinin combination have been found to directly inhibit activity of the two isoforms of cyclooxygenase (COX), constitutive (COX-1) and inducible (COX-2) which is expressed in response to inflammatory stimuli, without the adverse side effects attributed to synthetic COX inhibitors. The beta-sitosterol, caffeoylquinic acid, kaempferol, quercetin and zeatin combination, derived from the Moringa plant, is found to directly inhibit activity of COX isoforms, which makes this combination of ingredients naturally found in Moringa desirable in the treatment of inflammatory conditions, as well as for the symptoms of fever and pain.

In the present invention, it has been discovered that a mixture of the beta-sitosterol, caffeoylquinic acid, kaempferol, quercetin and zeatin, along with amino acids and essential fatty acids derived from a unique combination of Moringa plant components, in a single nutraceutical composition yields surprising health benefits. The efficacy of this zeatin-rich mixture derived naturally from the Moringa plant may be enhanced through the addition of selected natural juices, flavors, and/or colors which are believed to synergistically react with the natural combination of compounds from the Moringa plant. These natural flavors and colors can be derived from the juice of other fruits or vegetables. Furthermore, when placed in a nutraceutical beverage with fruit or vegetable juice, Moringa can be made palatable.

In one embodiment of the invention, the mixture from the Moringa plant can be complemented by the addition of one or more natural fruit or vegetable juices or juice extracts selected from, but not limited to, the following fruits or vegetables: apple, apricot, alfalfa, artichoke, asparagus, avocado, azerole, banana, beets, bilberry, blackberry, blueberry, bok choy, broccoli, cabbage, capsicum, cactus fruit, cane, cantaloupe, carrot, celery, cherimoya, cherry, coconut, corn, cranberry, cucumber, currants, durian, egg plant, ferijoa, figs, gooseberry, grapes, grapefruit, green barley, green beans, green lettuce, guava, ita palm, japonica, jujube, kale, kiwi, kumquat, legumes, lemon, lettuce, lime, loquat, oranges, mango, melon, mushrooms, nectarine, olives, papaya, parsley, passion fruit, pear, peas, persimmon, pineapple, plum, pomegranate, potato, pumpkin, prune, olive, quince, radish, raspberry, rhubarb, rice, rambutan, spinach, squash, starfruit, strawberry, sweet potato, tomato, tubors, ugli, voguanga, watermelon, xigua, yams and zucchini.

The formula for a composition of Moringa leaves, seeds and fruits is about 45-95% leaves, 9-40% seed cake and 1-10% fruit juice. It is preferred to have a composition of Moringa that is 65-85% leaves, 15-30% seed cake, and 1-8% fruitjuice. It is further preferred to have a composition of Moringa that is 70-82% leaves, 18-24% seed cake, and 2-6% fruit juice. When the Moringa composition is in the form of a beverage, the Moringa derived components comprise 5-40% of the total ingredients in a 5.5 fl oz (164.5 grams) daily can. The nutraceutical compositions of the present invention delivers therapeutic amounts of the natural compounds derived from the Moringa leaves (or from leaf extracts), fruit, fruit juice, seeds, seed oil, or seed cake. In one embodiment of the present invention, the mixture of Moringa leaves (or leaf extract) in an amount ranging from 35% to 90% with seed or seedcake represented in amounts between 5% and 45% and fruit or fruit juice ranging from the amounts 1% to 60% of the total weight of the nutraceutical Moringa composition in the nutraceutical beverage with any selected natural fruit or vegetable juice or juice extracts. This allows for a pleasant tasting beverage that includes virtually all of the advantageous compounds of the Moringa plant.

If the Moringa leaves, leaf extract, fruits, fruit juices, seeds, or seedcake are complemented with selected juices or juice extracts, then a liquid beverage is a convenient delivery form, but other delivery forms are efficacious as well and would simply require the use of powders, or other equivalent forms of the juices or juice extracts. The present composition can be delivered in any form known in the art, such as tablets, capsules, dispersions, oral sprays, inhalation sprays, tinctures, powders, solutions, suspensions, pastes, transdermal delivery system, and liquids. When the nutraceutical compositions of the present invention are formulated in liquid beverage form, which is presently preferred, the ratio of water to Moringa mixture and selected juices or juice extracts can be between 1:1 and 7: 1, preferably 3:1 or 4:1 and most preferably 5:1. It is also appreciated that, while not necessary, the nutraceutical Moringa compositions may utilize coloring agents to achieve a pleasing color.

The nutraceutical compositions of the present invention can be produced through large scale, economical operations. In one embodiment of the invented process, the Moringa leaves or leaf extract, fruits or fruit juice, and seeds or seedcake are picked and harvested and transported to a production facility. The fresh fruits, fruit pods, leaves and seeds can be kept at ambient air temperature during transportation, or frozen, depending on the need. The leaves are then shade dried in a hygienic area through ambient air, until only 5-8% of the leaf moisture remains, then ground into a powder through a cold commercial process grinding of the leaves. This cold commercial process protects the plant's antioxidant properties from oxygen, light and heat by cryogenically milling the leaves. The fruit pods are juiced to obtain the fruit juice by a commercial juicing or squeezing process. The seeds are harvested from the tree when they are mature and the oil is removed through cold press commercial practices using commercial squeezing or extraction methods that avoid heat, light and oxygen to prevent damaging the vitamins, minerals, antioxidants and anti-inflammatory properties found in the seed solids. The present invention utilizes the seed cake of the Moringa species, which is the byproduct of the commercial process of seed pressing in order to extract the seed oil. This commercial process keeps the vitamins, minerals and other active ingredients chemically undamaged during processing. The resulting mixture of Moringa leaves, leaf extract, fruits, fruit juice, seeds, seed oil or seedcake can then be further processed through mixing, and through the addition of one or more of the selected fruit or vegetable juices or extracts listed above. In the preferred embodiment of the beverage form of the invention, the selected juice extracts and purified water are then added to the mixture in accordance with the amounts, ranges and ratios specified above. The nutraceutical compositions can then be treated, bottled or packaged for distribution to consumers using a variety of methods known to those of ordinary skill in the art, such as flash pasteurization, sterilization, UHT sterilization, pressure sealing, freezing, freeze drying, irradiating, etc. Dehydrated and other forms of the nutraceutical compositions can also be prepared using standard techniques.

The effectiveness in improving general human health and wellness through the use of the nutraceutical Moringa compositions described herein is demonstrated from the following clinical examples, which are listed for illustrative purposes only, and are not meant to be limiting instances of therapeutic use. A therapeutic composition of the Moringa leaves, leaf extracts, fruits, fruit juice, seeds, seed oil, or seedcake mixture was prepared according to the embodiments described herein. Each subject ingested 5.5 fluid ounces of the beverage daily for a 30 day period. The following qualitative results were obtained:

### EXAMPLE 1

The subject was a 49-year-old male suffering from a history of chronic monthly migraine headaches, with incidence of at least one extreme migraine consisting of nausea, spotty vision, intense pain from light, sound or movement, that required medication, employment absences and black-out isolation. Each month also routinely brought several lesser migraine headaches characterized by typical migraine symptoms. In addition to migraine headaches, the subject also experienced sinus headaches that were both uncomfortable and painful. Prior to the study, the migraine headaches were treated with headache/migraine medication, sick leave from employment and black-out isolation from light, noise, and movement. After a regimen of taking the Moringa nutraceutical composition, the subject has not suffered a migraine headache of any kind.

### EXAMPLE 2

The subject was a 38-year-old male suffering from occasional seasonal cold viruses and the associated symptoms of muscle aches, runny nose, sinus pressure and headaches. After a regimen of the Moringa nutraceutical composition during cold symptoms, the subject reported a renewed sense of energy, improved mood, lessened muscle aches during the viral infection and a shortened duration of the virus infection, compared with other infections he experienced in the past.

### EXAMPLE 3

The subject was a female in her mid-fifties suffering from fibromyalgia or non-specific body and muscle aches, characterized by general fatigue and widespread pain in her muscles, ligaments and tendons. She previously unsuccessfully treated her symptoms by using over-the-counter and prescription pain medications, including synthetic COX-2 inhibitors. After a regimen of taking the Moringa nutraceutical composition, the subject experienced an elevated mood, more energy, less fatigue and less pain in her muscles, ligaments and tendons.

### EXAMPLE 4

The subject was a 48-year-old male suffering from chronic allergy-triggered asthma. Prior to taking the regimen of Moringa nutraceutical composition, the subject treated asthma with Pulmacort and Albuterol steroid medications, and experienced common side effects. After the Moringa nutraceutical composition regimen, the subject reduced Albuterol use by an estimated 70% and decreased its use from several times per day to only a couple of times a week. The subject believes that the Moringa nutraceutical composition has reduced his sensitivity to allergen triggers that bring on asthmatic episodes.

### EXAMPLE 5

The subject was a 51-year-old male suffering from high blood pressure averaging around 165/110-115. After 30 days on a regimen of the Moringa nutraceutical composition, the subject felt more energy, less fatigue and recorded an average blood pressure reading of 140/90, or a drop of 25 points on both the systolic and diastolic pressures.

### EXAMPLE 6

The subject was a 48-year-old female suffering from Type I diabetes for the past seven (7) years. Prior to a regimen of Moringa nutraceutical composition, the subject treated the diabetes with insulin shots averaging three times per day, and tested within the normal range only 75% of the time during the blood glucose tests. After 30 days of drinking a regimen of the Moringa nutraceutical composition, the subject experienced improvement in her diabetes, and now she averages only two shots of insulin per day and requires 50% less of the fast acting insulin and 25% less of the slow acting insulin. After this regimen and continued use of the Moringa nutraceutical composition, the subject registers normal blood glucose levels 90% of the time, in all tests.

### EXAMPLE 7

The subject was a male in his mid-fifties who used reading glasses during the course of the work day on a near constant basis. After a regimen of taking the Moringa nutraceutical composition, the subject reported improved eyesight that enabled him to see more clearly, and did not require the use of his reading glasses nearly as often as prior to the regimen.

### EXAMPLE 8

The subject was a 28-year-old female suffering from a dry skin condition that required brand name conditioners and moisturizers in order to keep her skin from being dry and flaky. After a regimen of the Moringa nutraceutical composition, the subject experienced smoother, softer and more elastic skin that enabled her to discontinue the expensive moisturizers and conditioners.

### EXAMPLE 9

The subject was a 48-year-old male with arthritic knees and joint deterioration due to injury and age. Subject treated the pain associated with the conditions with acetaminophen and ibuprofen (pain relievers). Since taking the Moringa nutraceutical composition, the subject has noticed that the pain is lessened in the knee joints and self-medication is lessened significantly from three to four ibuprofens every six hours on a daily basis to now taking this medication only during severe weather changes.

### EXAMPLE 10

The subject was a 53-year-old male suffering from acid reflux disease, poor digestion, type II diabetes and stress related depressed moods. After a regimen of the Moringa nutraceutical composition, the acid reflux symptoms were eliminated, digestion was dramatically improved and the subject noticed that his diabetes medicine was faster acting. In addition, his ability to handle stressful situations was improved, due to his overall sense of well-being.

### EXAMPLE 11

The subject was a 36-year-old male physically-fit athlete. After a regimen of the Moringa nutraceutical composition, the subject experienced increased energy levels and endurance during strenuous work-outs, and a fuller and darker color head of hair; along with smoother skin.

### EXAMPLE 12

The subject was a 54-year-old male with high blood pressure and type II diabetes, controlled by medication. Before the regimen of Moringa nutraceutical composition, the subject's blood glucose level was as high as 278 and his blood pressure ranged from 168/92 to 170/98. After taking the Moringa nutraceutical composition and while still consuming the product, the blood glucose levels were from 70 to 124 and the blood pressure readings are now around 122/80. The subject reports sleeping better at night and feeling healthier.

### EXAMPLE 13

The subject was a 50-year-old male desiring to lose a few pounds and maintain a healthy lifestyle. After a regimen of the Moringa nutraceutical composition, the subject lost one inch from his waist and three pounds in body weight. The subject reported his appetite was decreased, enabling him to wisely choose the foods he ate, and that the quantity of food consumed during meals was reduced. The subject reported increased energy and a sense of well-being.

### EXAMPLE 14

The subject was a 49-year-old male suffering for the last 10 years from depression. His condition was treated with the prescription medication, Zoloft, which helped manage the depression, but the depression was still evident. After the regimen of the Moringa nutraceutical composition, the subject felt happier and healthier and has stopped taking the expensive prescriptions. The subject reports no symptoms of depression after the Moringa nutraceutical composition regimen.

### EXAMPLE 15

The subject was a female in her mid-fifties. After the regimen of the Moringa nutraceutical composition, the subject noticed her head of hair was changing back to her natural brown color resulting in fewer gray hairs and a fuller, healthier head of hair. In addition, the subject reported smoother, more elastic skin and that her wrinkles and fine lines were less noticeable than before the Moringa regimen.

Thus, there is disclosed new nutraceutical compositions and methods for using the same. The present invention may be embodied in other specific forms without departing from its spirit or essential characteristics. The described embodiments are to be considered in all respects only as illustrative and not restrictive.

1.-72. (canceled)

73. A nutraceutical Moringa composition comprising:
leaves from a Moringa plant; and
a portion of the seeds from a Moringa plant other than the oil.

74. The composition of claim 73, wherein the composition further comprises a portion of the fruit from a Moringa plant other than the seeds.

75. The composition of claim 73, wherein the composition further comprises a fruit or vegetable other than the Moringa plant.

76. The composition of claim 73, wherein the leaves and seeds are ground.

77. The composition of claim 73, wherein the composition is formed into at least one of the group consisting of a syrup, a drink mix, a beverage, a tablet, a capsule, a gel, and a spray.

78. The composition of claim 73, wherein the composition further comprises water.

79. The composition of claim 73, wherein the leaves from the Moringa plant comprise both leaves harvested from a Moringa plant between 2 months and 10 months of age and leaves harvested from a Moringa plant between 1 year and 25 years of age.

80. The composition of claim 73, wherein a percentage by weight of the leaves of the Moringa plant is approximately 55% to 85% of the total weight of the Moringa plant components.

81. The composition of claim 73, wherein a percentage by weight of the leaves of the Moringa plant is approximately 70% to 80% of the total weight of the Moringa plant components.

82. The composition of claim 73, wherein a percentage by weight of the seeds of the Moringa plant is approximately 15% to 30% of the total weight of the Moringa plant components.

83. The composition of claim 73, wherein a percentage by weight of the seeds of the Moringa plant is approximately 17% to 27% of the total weight of the Moringa plant components.

84. The composition of claim 74, wherein a percentage by weight of the fruit of the Moringa plant is approximately 3% to 8% of the total weight of the Moringa plant components.

85. A nutraceutical Moringa composition comprising:
at least two portions of a Moringa plant selected from the group consisting of: the leaves from a Moringa plant, a portion of the seeds from a Moringa plant other than the oil, and a portion of the fruit of a Moringa plant other than the seeds.

86. The composition of claim 85, wherein a percentage by weight of the leaves of the Moringa plant is approximately 70% to 80% of the total weight of the Moringa plant components, a percentage by weight of the seeds of the Moringa plant is approximately 17% to 27% of the total weight of the Moringa plant components, and a percentage by weight of the fruit of the Moringa plant is approximately 0% to 8% of the total weight of the Moringa plant components.

87. The composition of claim 85, wherein the Moringa plant components are ground and formed into a beverage.

88. The composition of claim 85, wherein the composition further comprises a fruit or vegetable other than the Moringa plant.

89. A method of preparing a nutraceutical composition comprising:
grinding leaves from a Moringa plant;
grinding seeds from a Moringa plant including a portion of the seeds other than the oil; and
combining the leaves from a Moringa plant and the seeds from a Moringa plant including a portion of the seeds other than the oil into a nutraceutical composition.

90. The method of claim 89, wherein the method further comprises combining a fruit or vegetable component not from a Moringa plant into said nutraceutical composition.

91. The method of claim 89, wherein the method further comprises combining a portion of the fruit from a Moringa plant other than the seed into said nutraceutical composition.

92. The method of claim 89, wherein the method comprises forming said nutraceutical composition into a beverage.

93. The method of claim 89, wherein the method comprises forming said nutraceutical composition such that a percentage by weight of the leaves of the Moringa plant is approximately 70% to 80% of the total weight of the Moringa plant components and such that a percentage by weight of the seeds of the Moringa plant is approximately 17% to 27% of the total weight of the Moringa plant components.
